(19) United States
(12) Patent Application Publication
(10) Pub. No.: US 2007/0264704 A1
(43) Pub. Date: Nov. 15, 2007

(54) Bio-filter with low density media and toroidal media stirring configuration

(76) Inventor: J. Wayne Van Toever, Bristol, TN (US)

Correspondence Address: SIMPSON & SIMPSON, PLLC, 5555 MAIN STREET, WILLIAMSVILLE, NY, 14221-5406, US

(21) Appl. No.: 11/729,725

(22) Filed: Mar. 29, 2007

Related U.S. Application Data
(63) Continuation-in-part of application No. PCT/CA05/01445, filed on Sep. 23, 2005.
(60) Provisional application No. 60/614,222, filed on Sep. 29, 2004.

(51) Int. Cl. C02F 3/02; C12M 1/06

(52) U.S. Cl. 435/262; 210/170.02; 210/170.08; 210/196; 435/305.1; 435/305.2

(57) ABSTRACT

A bio-reactor having a downflow self-cleaning configuration includes a liquid deflecting means generally concentric with a cylindrical tank. The liquid deflecting means is located just below the surface of liquid in the tank and is configured to direct liquid radially outwardly towards an inner surface of the tank. The liquid flows downwardly adjacent the inner surface, then inwardly toward the center of the tank and upwardly adjacent the center of the tank. The liquid circulates in a toroidal configuration to stir buoyant media pellets within the tank. A variation of the design incorporates upwelling injected diffused air to induce the same toroidal circulation pattern.

### CROSS-REFERENCE TO RELATED APPLICATIONS

This application is a Continuation-in-Part of International Application No. PCT/CA2005/001445, filed on Sep. 23, 2005, which claims the benefit of U.S. Provisional Patent Application No. 60/614,222, filed on Sep. 29, 2004, which applications are incorporated herein by reference.

### FIELD OF THE INVENTION

The invention relates to bio-filters (alternately known as bio-reactors), which are used to culture a wide variety of micro-organisms, which digest dissolved and fine particulate organic compounds from wastewater or are used to produce specific end products such as pharmaceuticals. The class of bio-reactors in which the invention is included use a stirred bed of small, low density (floating) plastic particles with an optimized surface structure which shelters an optimal thickness of bio-film.

### BACKGROUND OF THE INVENTION

US patents to Van Toever U.S. Pat. Nos. 5,055,186, granted Oct. 8, 1991; 5,593,574, granted Jan. 14, 1997; and 6,617,155, granted Sep. 9, 2003 describe the evolution of a bio-reactor which utilizes a floating bed of low density plastic media particles on which bio-film is cultured. The objective is to culture as much active, effective bio-film as possible per unit volume of bio-reactor. To be successful this requires:
providing media with optimal surface structure for culture and shelter of the optimal thickness of bio-film;
design of media particles which maximize the quantity of bio-film per unit volume of filter, i.e. provides the maximum achievable surface area per unit volume of media;
providing a method of shearing excess bio-film growth from the media to maintain the optimal thickness for nutrient and oxygen transfer to the base of the bio-film;
providing an energy efficient method to move uniformly the filtrate, which contains the nutrients and oxygen required for growth of the micro-organisms, to every media particle in the filter bed;
designing a robust, maintenance free design for the bio-reactor to accomplish the above;
design of a particulate collection and removal system.

Efforts to develop a more efficient and more easily scalable bio-reactor have led to the development of a new design which is mechanically much simpler than previous designs, more energy efficient and easily scalable to large sizes. The design concepts are similar to the previous designs and the filter utilizes the same filter media described in the previous designs.

The most recent patent, Van Toever U.S. Pat. No. 6,617,155, “Fluidized Radial Flow Bio-reactor utilizing pelleted Media” describes a low density media bio-reactor with a rotating filtrate distribution manifold. Although described as a “Fluidized Filter” this was a technical error in language as the filter actually is a “Stirred Filter” design which is considered to be a different functional category as discussed further below. Most of the filters that have been built with this design utilize, for example, a low speed (1 rpm) gear motor to rotate the manifold. While the media stirring and biological performance of these filters has been effective, the mechanical design requires significant maintenance especially when used in corrosive wastewater applications. Industrial wastewater treatment environments are generally warm, high humidity, corrosive environments and the filters must run continuously year round. The gearmotors and components are also relatively costly components. Although relatively large filters (5 m³ media volume) have been constructed, the mechanical complexity of the design imposes practical limits on scale-up potential. To treat large industrial or municipal wastewater flows, much larger filters must be constructed. Because of this, recent research efforts have focused on developing a simpler, more efficient and dependable design for uniformly injecting the filtrate and for stirring the media bed.

Shimodaira et al. U.S. Pat. No. 4,256,573, describes a low density media bio-filter which is based on fluidizing the media bed with a downward flow of filtrate. Various configurations of inlet distribution manifolds or perforated distribution plates are described, which attempt to uniformly distribute the filtrate over the media bed surface in a downward direction, to expand and fluidize the media bed.

As discussed in Shimodaira et al. U.S. Pat. No. 4,256,573, bio-filters or bio-reactors using fine particulate carriers (media) fall into three categories including; fixed beds, fluidized beds and completely mixed or stirred beds. The distinguishing features of each category are described below.

Fixed Beds

Fixed beds are constructed of static beds of particulate media over which the filtrate is uniformly distributed. Bio-film growth and waste particles in the filtrate rapidly clog the spaces between the particles which causes channeling of flow and ineffective filtration. Complex backwash systems are therefore required to clean the beds which shear most of the bio-film along with the solids. The bio-film then has to re-establish itself so the bio-filtration process never reaches an optimal equilibrium phase. Fixed bed filters are an old ineffective design and have been largely replaced by either stirred or fluidized designs. They can be used effectively for mechanical particulate removal but are not effective bio-filters.

Fluidized Beds

There are two categories of fluidized beds. High Density Media filters use media such as sand with a density greater than the filtrate and they are fluidized in an upflow direction. Low Density Media filters use media such as plastic with a density less than the filtrate and they are fluidized in a downward direction.

The filters are usually tall and narrow to reduce pumping flow rates required to achieve sufficient linear velocity to fluidize the media although this increases the pumping head and energy consumption requirements. The filtrate is uniformly distributed over the media surface in this design.

a-High Density Media

Fluidized beds with high density media such as sand (denser than filtrate) use an upflow design. Filtrate usually enters the filter from the bottom through a perforated distribution plate or nozzles which cause a uniform upward flow over the cross section of the filter. The velocity is selected to expand the bed of media upwards so that the particles hover in place. If the velocity is too high, the media will wash out of the top of the filter. As the bio-film accumulates on the media, the particle density decreases because bio-film (density approximately 1.1 relative to water) is less dense than the high density media particles. The bed gradually expands and the velocity must be adjusted or devices must be installed to shear off excess media or the media will wash out of the top of the filter. The use of high density particles such as sand requires greater pumping head and energy consumption to fluidize the media relative to low density fluidized media filters which generally use media with densities close to but less than that of the filtrate.

b-Low Density Media

Low density designs use particulate media with a density less than the filtrate so that they float. In the Shimodaira et al. U.S. Pat. No. 4,256,573, design, the filtrate is distributed uniformly over the filter media bed surface and flows downward which counteracts the buoyancy of the low density plastic particles. The bed expands downward and the particles fluidize. This design requires less energy for fluidization since the density of the media is very close to that of the filtrate. The fluidization action is relatively gentle, and therefore devices such as mechanical stirrers must be installed to shear excess bio-film, which accumulates as the bio-film grows. Since the density of the bio-film is greater than the filtrate and low density media, as it accumulates the media sinks and can wash out the base of the filter. Filters of this design can use screens to maintain the media in the filter, however they are prone to fouling with bio-film growth.

Stirred Beds

Stirred beds consist of a vessel of media which can have a density either greater or less than the filtrate. A mechanical stirring device, an aeration system or a pumped filtrate distribution system can be used to stir the media bed. The stirring action keeps the media in suspension and constant motion and ensures uniform contact between the bio-film on the particulates and filtrate. If the media used is similar in density to the filtrate, the media can be stirred with relatively little energy. The movement of the media particles in the filter media bed is relatively random. The stirring action usually causes the media to distribute uniformly throughout the filter vessel. Screens are usually used to prevent the media from washing out of the filter and the screen opening size must obviously be less than the particle size to retain them. With use of small sized media particulates or pellets to maximize surface area per unit volume, the required fine screens are prone to plugging from bio-film growth and cleaning systems usually must be employed.

The previous and this new Van Toever filter designs are of the stirred media category although they have been erroneously referred to in previous patents, as noted above, as being “fluidized” designs. The filter described in U.S. Pat. No. 5,055,186 is an air stirred filter with media retention screens. The subsequent designs including U.S. Pat. Nos. 5,593,574 and 6,617,155 and the new invention described herein are also of the Stirred Filter category. They also feature stirred floating low density media beds which occupy the upper zone of the filter vessel. There is a media free filtrate zone below the media bed and the filtrate flows out of the filter at the base of this zone so that media retention screens with their inherent plugging problems are not required in these designs. Additionally the filters feature a unique cone bottomed base where solids can be separated from the filtrate flow, stored and subsequently flushed from the filter out a separate drain line.

### SUMMARY OF THE INVENTION

The new bio-reactor has many of the advantageous features of the previous Van Toever designs including the downflow self-cleaning design with solid waste collection in the cone bottomed base, no inlet or outlet screens to foul and energy efficient design. The new design, however has a much simpler, more versatile and energy efficient filtrate injection and media stirring system which can easily be scaled up for large applications.

As noted, the new filter design is of the “Stirred” media design category and the media can be stirred either with injected filtrate or air injection. With the “injected filtrate” design the media is stirred by injecting filtrate through a novel fluid deflecting means design which induces a unique toroidal (donut) shaped stirring configuration. The “air injection” design uses upwelling air injected through an air lift pump or air diffusers to create the toroidal circulation flow. There are several advantages with the new design, which are described below.

The bio-filter or bio-reactor uses the same floating low density plastic media with an enhanced surface to shelter the optimal thickness of bio-film as in previous designs. The bed of media is stirred by the flow of pumped filtrate which induces a toroidal shaped circulation configuration. Other low density plastic media designs can also be utilized including commercially available products.

Although several configurations and embodiments are outlined hereafter, the basic configuration and operation of all options are the same with respect to the toroidal configuration of the media being stirred.

### BRIEF DESCRIPTION OF THE DRAWINGS

The nature and mode of operation of the present invention will now be more fully described in the following detailed description of the invention in view of the accompanying drawing figures, in which:

FIG. 1 is a schematic side perspective view of a device according to the present invention;

FIG. 2 is a schematic side perspective view of an alternate embodiment according to the present invention;

FIG. 3 is a schematic side perspective view of further alternate embodiment according to the present invention;

FIG. 4 is a cross-sectional view of the embodiment illustrated in FIG. 3;

FIG. 5 is an enlarged cross-sectional side view of an axial flow pump/aerator according to the embodiment illustrated in FIG. 3;

FIG. 6 is a sectional view taken along 6-6 of FIG. 5;

FIG. 7 is a schematic cross-sectional side view of a modification of the embodiment illustrated in FIG. 3;

FIG. 8 is a schematic cross-sectional side view of a floating bio-reactor or bio-filter similar to the FIG. 3 embodiment for use in wastewater treatment reservoirs or ponds;

FIG. 9 is an illustration of a bio-filter with internally re-circulated liquid and an air lift pump;

FIG. 9a is an enlarged detailed view of the air pump shown in FIG. 9;

FIG. 10 is a sectional view along line 10-10 of FIG. 9;

FIG. 11 is a cross-sectional side view of the embodiment illustrated in FIG. 1 in association with an aquaculture tank;

FIG. 12 is a cross-sectional side view of the embodiment of FIG. 2 in association with an aquaculture tank;

FIG. 13 is a cross-sectional side view of a further embodiment of the inventive bio-filter with different air media stirring;

FIG. 13a is an enlarged view of a portion of FIG. 13 as shown; and,

FIG. 14 is a sectional view through line 14-14 of FIG. 13.

### DETAILED DESCRIPTION OF THE INVENTION

At the outset, it should be appreciated that like drawing numbers on different drawing views identify identical, or functionally similar, structural elements of the invention. While the present invention is described with respect to what is presently considered to be the preferred aspects, it is to be understood that the invention as claimed is not limited to the disclosed aspects.

Furthermore, it is understood that this invention is not limited to the particular methodology, materials and modifications described and as such may, of course, vary. It is also understood that the terminology used herein is for the purpose of describing particular aspects only, and is not intended to limit the scope of the present invention, which is limited only by the appended claims.

Unless defined otherwise, all technical and scientific terms used herein have the same meaning as commonly understood to one of ordinary skill in the art to which this invention belongs. Although any methods, devices or materials similar or equivalent to those described herein can be used in the practice or testing of the invention, the preferred methods, devices, and materials are now described.

(1) External Pumped Recirculation Loop with Low Wastewater Flow

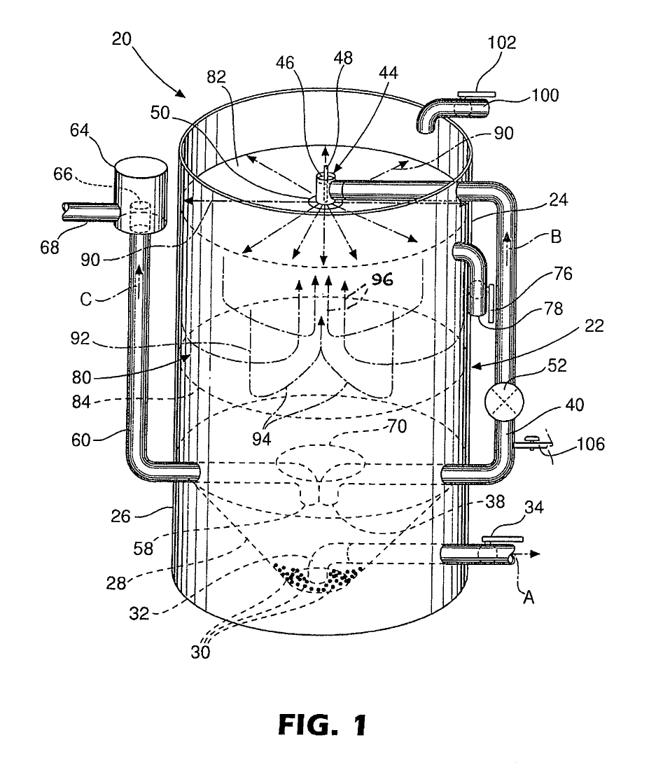

Turning to FIG. 1 there is shown schematically inside perspective view a bio-filter or reactor 20 including tank 22 with peripheral wall 24 and support base 26 within which is coned shaped particulate collector 28 for settled particulate 30. Settled particulate outlet manifold 32 and valve 34 allow for periodic removal of particulates from collector 28 as shown by arrow A. Within tank 22, spaced vertically above particulate collector 28 is inlet 38 of recirculated filtrate manifold 40. Manifold 40 extends radially outwardly, externally of the tank wall 24, vertically, and then back through the tank wall 24 to be in flow communication with filtrate recirculation inlet nozzle 44, i.e., a liquid deflecting means. Inlet 38, manifold 40 and nozzle 44 define a filtrate recirculation loop. Filtrate recirculation nozzle 44 is cylindrical and has an upper plug plate 46 with a threaded hole which is adapted to threadingly engaged support shaft 48 of adjustable distribution disk 50. A flow gap 52 is defined between the bottom of cylindrical nozzle 40 and disk 50.

Filtrate recirculation pump 54 is located in manifold 40 to cause filtrate to flow in the filtration recirculation loop as shown by arrow B.

Adjacent filtrate recirculation inlet 38 is treated filtrate effluent outlet 58 which is in flow communication with manifold 60 which leads to a filtrate/media level control chamber 64 with adjustable level control pipe 66. Treated filtrate exits the system via outlet 68. Flow through manifold 60 is shown by arrow C. Attached to and above recirculate filtrate manifold inlet 38 and treated filtrate effluent manifold outlet 58 is baffle plate 70 to prevent particulate material and possibly media pellets from being sucked into the inlet 38 or outlet 58. Plate 70 can be slightly conical so that particulate material flows outwardly and downwardly away from the manifold inlet 38 and outlet 58.

Further, pipe 78 with valve 76 permits valve controlled removal of media (pellets) as desired to analyze the bio-film on the media.

As shown in FIG. 1, media bed 80, preferably plastic pellets as described in my previous patents, the disclosures of which are incorporated herein, has upper surface or level 82 which is the upper level of the wastewater to be treated and a lower level 84, the media being buoyant and floating in the tank in the water to be treated.

In operation, filtrate recirculated through the filtrate recirculation loop by pump 52, exits generally horizontally and radially from the recirculated filtrate nozzle 44 because it impinges on disk 50 and flows as shown by arrows 90 radially outwardly toward the inside of tank wall 24. Flow continues downwardly along the peripheral wall 24 as shown by arrows 92, then horizontally inwardly at the lower level 84 of the media bed as designated by arrow 94 and then vertically adjacent the axis of the tank as shown by arrows 96. The flow agitates and stirs the media pellets sufficiently to cause scraping of the media pellets against one another to remove excess bio-film particulate which gravitates to the collector 28 and can be periodically removed through manifold 32.

It will be appreciated that as treated liquid is removed from outlet 68, make up untreated wastewater is allowed to flow into the tank 22 through wastewater inlet 100 and this flow can be controlled automatically by a level control sensor operating valve 102.

Valved oxygen line 106 associated with manifold 40 permits oxygen to be added to the recirculated filtrate loop to assist in promoting growth of film on the media.

(2) Non Recirculated—High WasteWater Flow

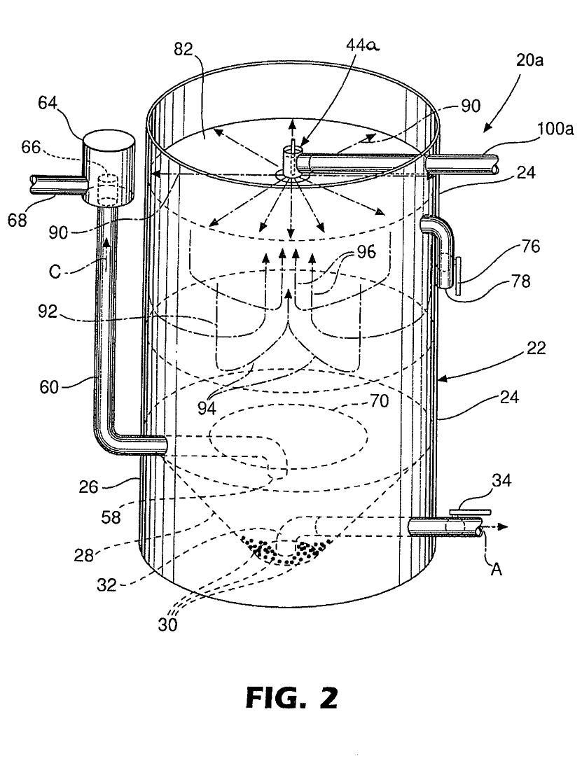

FIG. 2 shows a modified reactor embodiment 20a wherein like elements and structures bear the same numerical reference as they do in FIG. 1. Whereas FIG. 1 depicts a filtrate recirculation loop with low wastewater inlet flow, FIG. 2 relates to a non-recirculated flow system which can be used for high wastewater flow. Wastewater is directed straight to the inlet nozzle 40a, a liquid deflecting means, via manifold 100a and there is no filtration recirculation loop as shown in FIG. 1.

(3) Internal Recirculated—Low WasteWater Flow

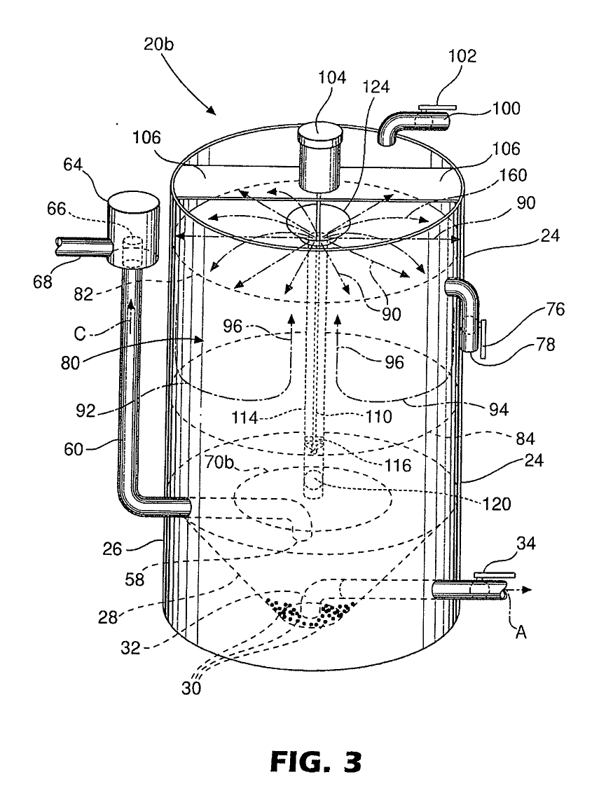

FIGS. 3, 4, 5 and 6 show a further alternate reactor embodiment 20b having an internal recirculating flow for particular use with low wastewater inlet flow. Again elements and features similar to those in FIG. 1 bear the same numerical references.

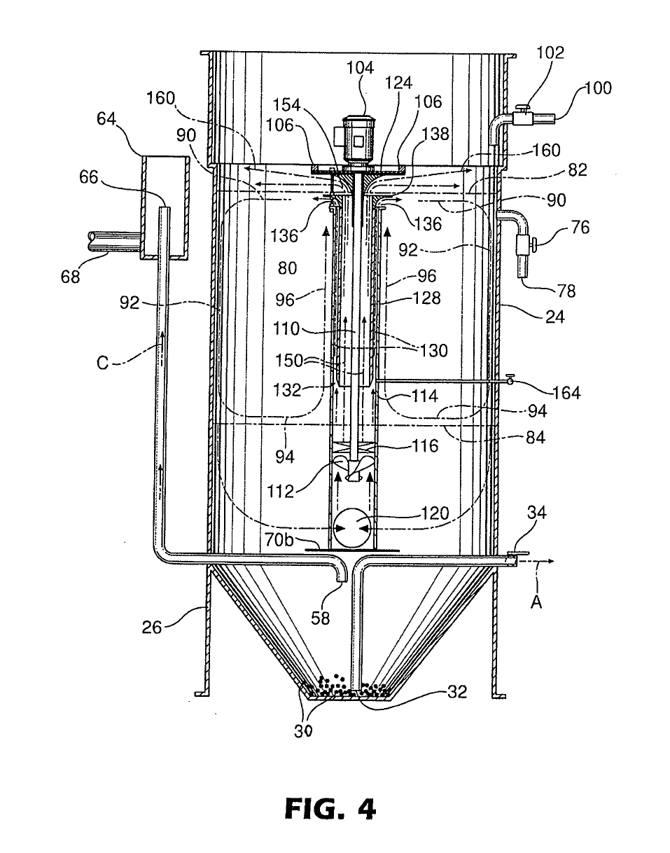

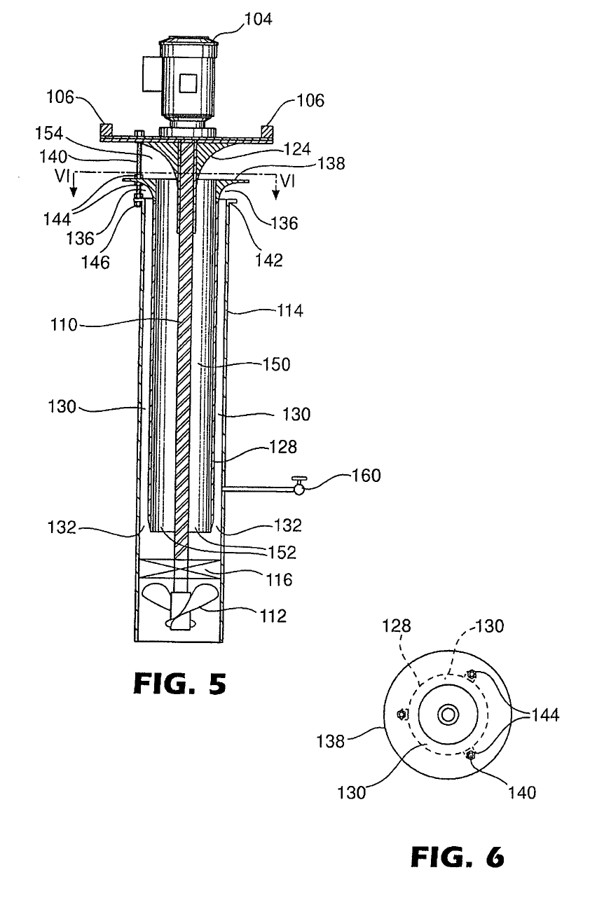

FIG. 3 is a schematic side perspective view whereas FIG. 4 is a schematic side sectional view and FIG. 5 is an enlarged portion of FIG. 4. Pump/aerator electric motor 104 is supported above the water level by cross support frame 106. Pump drive shaft 110 passes rotatably through support frame 106 and extends downwardly to drive impeller 112. Impeller 112 is located in pump section manifold 114 and is supported in manifold 114 by bearing support structure 116. Manifold 114 has at its lower end filtrate inlet 120, and baffle 70b is secured to the bottom of the manifold 114. Inlet 120 is located in the peripheral side of manifold 114 just above baffle 70b. Below motor 104 is deflection disk 124, a liquid deflecting means which is contoured as shown in FIGS. 4 and 5 to deflect filtrate pumped upwardly within manifold 114 and redirect it radially outwardly. Disk 124 is stationary and is shown attached to support frame 106 in FIGS. 4 and 5, pump shaft 110 rotatably extending therethrough. Within manifold 114, as best shown in FIGS. 4 and 5, is a concentrate flow channel divider 128, divider 128 being concentric within manifold 114 and defining a flow path 130 including inlet 132 therebetween. At the upper end of divider 128 is a flow channel 136 defined by peripheral flange 138 which is contoured as shown in FIG. 5 to also deflect pumped liquid radially outwardly but below that deflected by disk 124.

As will be further noted in FIGS. 5 and 6, FIG. 6 being a sectional view through line 6-6 of FIG. 5, flange 138 is adjustably supported on threaded bolts 140 which are peripherally spaced about flange 138, bolts 140 being connected to flange 142 of divider 128 with nuts 146 and through upper disk 124 to support frame 106. Nuts 144 position and lock flange 138 in a selected position.

Accordingly, two flow channels are defined, a central aeration flow channel 150 and a concentric flow channel or path 130 with lower inlets 152 and 132, respectively, and upper outlets 154 and 136, respectively.

The embodiments of FIGS. 3 to 6 provide two upward flow passages 130 and 150, flow passage 150 and outlet 154 providing radially outwardly sprayed filtrate 160 which is aerated and falls back into the filtrate in the tank. Flow path 130 defined between the concentric divider 128 and the wall manifold 114 exists subsurface of the upper level 82 of filtrate and is the flow which creates the toroidal flow pattern of filtrate and media pellets as shown by arrows 90, 92, 94 and 96. Valved conduit 164 permits the injection of oxygen into the flow path 130 as desired to promote bio-film growth.

(4) Internal Recirculated System with Submersible Pump and No Particulate Collection

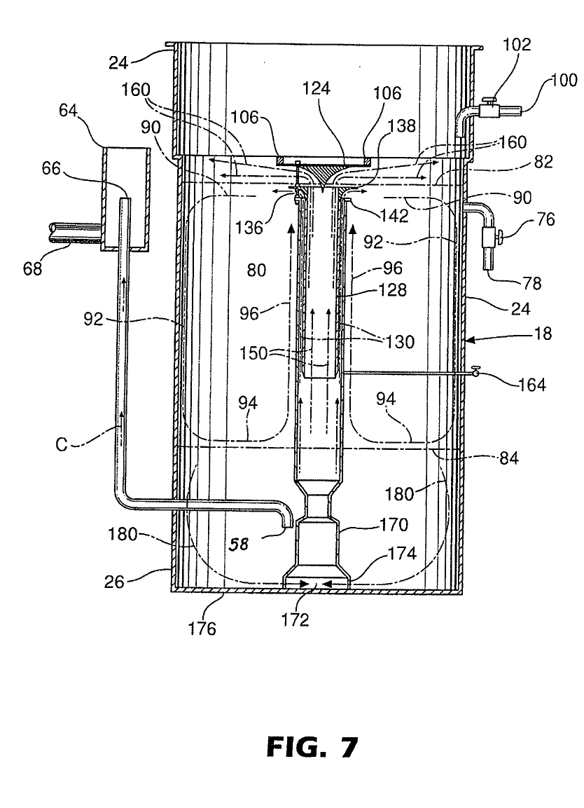

FIG. 7 shows schematically an embodiment similar to that in FIGS. 3, 4, 5 and 6 but with an internal submersible pump 170 for internal recirculation of filtrate and no solids or particulate collection. Inlet housing 172 to pump 170 is screened by peripheral screen 174 between housing 152 and floor 176 of tank 18, recirculation flow of filtrate is shown by arrows 180 and sprayed filtrate 164 whereas the toroidal movement of the media is shown by arrows 90, 92, 94 and 96.

(5) Floating Bio-Filter for Use in Wastewater Reservoirs

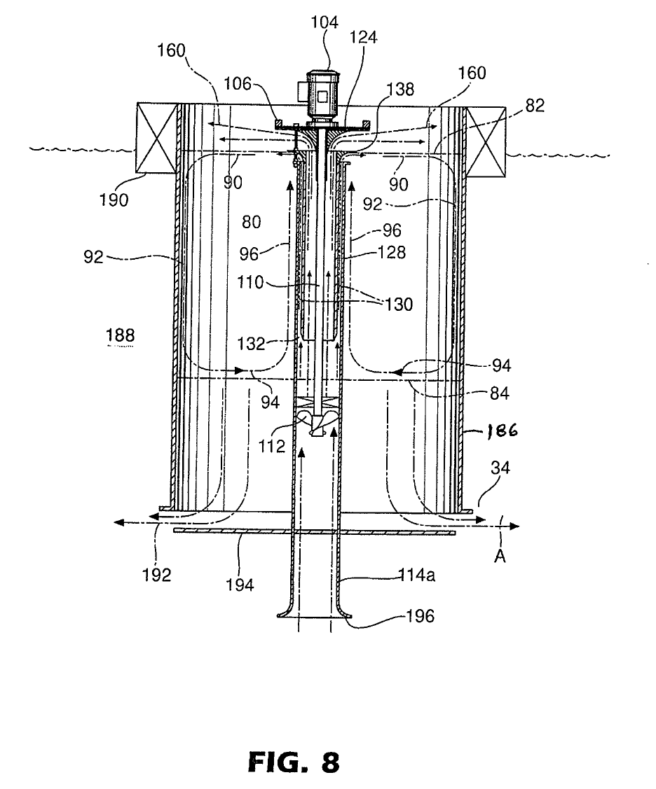

FIG. 8 shows a further embodiment, similar to that in FIGS. 3-6, but adapted for floating the bio-filter for use in wastewater treatment reservoirs or ponds 188. Floating collar 190 supports bio-filter 20a including pump motor 104, impeller 112, and pump section manifold 114a. Manifold 114a extends deeper into the reservoir water. Filtrate which does not flow with the movement of pellet media along arrow 94 to flow from within the tank wall will flow outward back into the pond 188 through peripheral gap 192 defined by the bottom 194 of tank 186 and plate 194 which is secured to manifold 114a. Manifold 114a has an open bell shaped bottom inlet 196.

(6) Bio-Filter—Internally Recirculated with Air Lift Pump

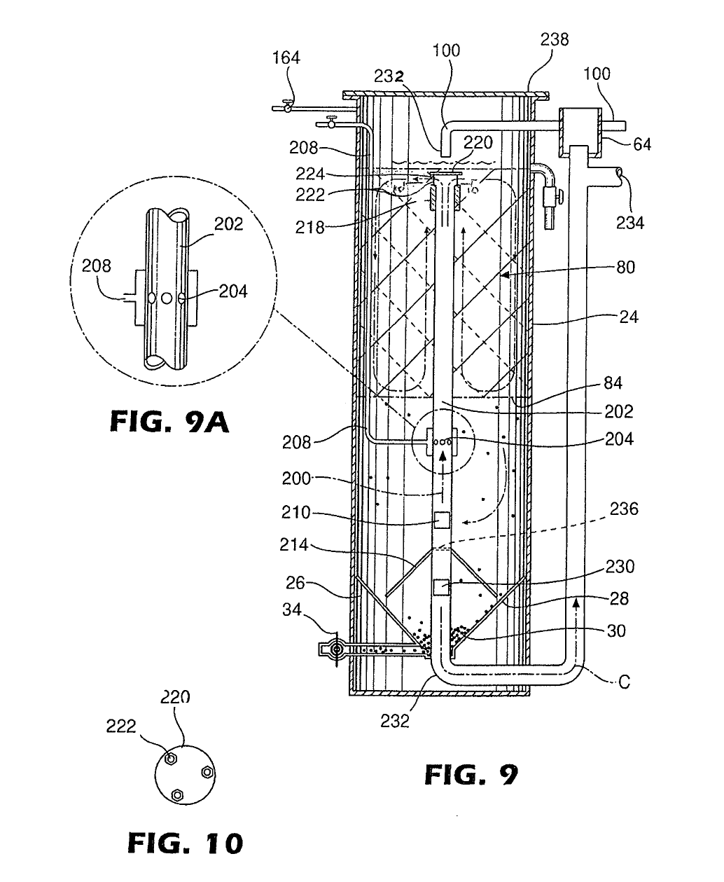

FIGS. 9, 9a and 10 illustrate a further modified embodiment of the bio-filter or bio-reactor with recirculation of the filtrate using an air lift pump 200. Recirculation manifold 202 has peripherally spaced apertures or openings 204 therein and air lift pump collar 206 is secured about the periphery of the manifold 200 to define an air chamber which is in flow communication with air supply conduit 208 as also shown enlarged in FIG. 9A. Below air lift pump 200 is the air lift pump inlet 210 and below it is conical baffle 214 to deflect particulate material scoured from pellet media 80 which collects in collector 28 and be periodically removed through valved manifold 34.

At the upper end of manifold 202 is upper flange 218 and flow distribution disk or plate 220, a liquid deflecting means, which directs subsurface flow of filtrate outwardly as in other embodiments herein, through outlet 224. Plate 220 is supported, by the upper end flange 218 of manifold 202 which flange is structurally supported within tank 24 by means not shown, the disk or plate 200 being adjustably supported by peripherally spaced threaded rods 222 associated with upper flange 218 of manifold 202 as shown in sectional view FIG. 10. Nozzle outlet 224 is defined between the bottom of plate 220 and the upper surface of flange 218. Disk 220 is mounted subsurface of the filtrate so that filtrate impinging on it will be deflected outwardly as in other embodiments. Plate 220 may be contoured as in other embodiments. This embodiment also shows cover 238 detachably secured to the top of the tank which would enable pure oxygen to be injected through conduit 164. The cover defines a splash zone above the media bed. Although shown in this embodiment, a cover could be used with those embodiments with splash aeration sprays such as shown in FIG. 3, with a relocation of oxygen conduit 164 to the sealed splash zone.

Beneath baffle 214 is effluent outlet 230 in flow communication with manifold 232 leading to treated water outlet 234. It will be appreciated that recirculation manifold 200 and effluent manifold 230 are separated by plate 236 secured within manifold 202 in this embodiment.

Wastewater inlet 100 may be along the side of the tank wall 24 or as shown in this embodiment where it is directed through outlet 238 into plate 220 to splash outwardly for aeration purposes.

The embodiment of FIG. 9 is shown without all the support for the piping, manifolds, and the like for the sake of simplicity, the important aspect being that the flow of filtrate and media is toroidal as in other embodiments.

(7) Aquaculture System with External Pump

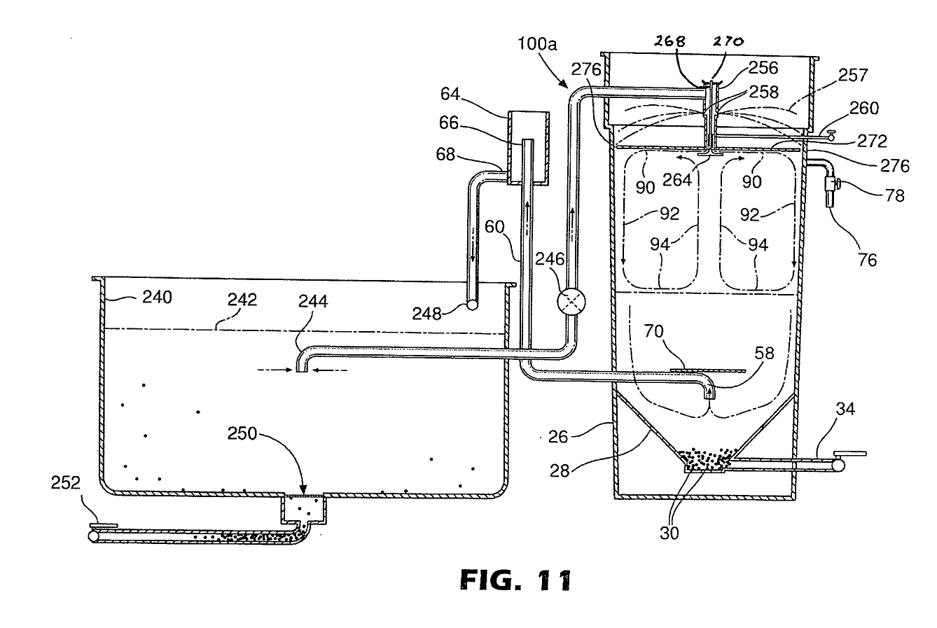

FIG. 11 is a schematic illustration of the embodiment of FIG. 2 in combination with an aquaculture tank 240.

The aquaculture tank 240 with water level 242 has screened wastewater intake 244 connected to bio-filter wastewater intake manifold 100a through pump 246 with treated water pipe 68 leading back to tank 240 and directed by nozzle 248 to create a rotational motion to the water in fish tank 240 which is well known in the art. Fish tank 240 has a particulate screened outlet construction 250 with valve 252.

In the embodiment of FIG. 11 however, the inlet wastewater is divided in manifold 256 so that some of the wastewater is sprayed outwardly 257 through aperture 258 to facilitate aeration. Oxygen may be added to the inflow through line 260. Contoured disk 264, a liquid deflecting means, is slightly subsurface of the water level 82 in bio-filter tank 218 and causes the flow of wastewater to move radially outwardly as in the earlier embodiments to create the toroidal movement of media in the filtrate.

Disk 264 is supported by threaded rod 268 which is adjustable at 270 to optimize toroidal flow.

Plate 272 is attached to the bottom of manifold 256 so that sprayed wastewater 257 is forced to enter the tank at peripheral gaps 276 and not disturb the outward stirring flow 90 of media and filtrate. Treated filtrate exits from the tank via outlet 58 to which baffle plate 70 is attached as in other embodiments. Outlet 58 leads to manifold 60 as noted above.

(8) Aquaculture—Internal Pump

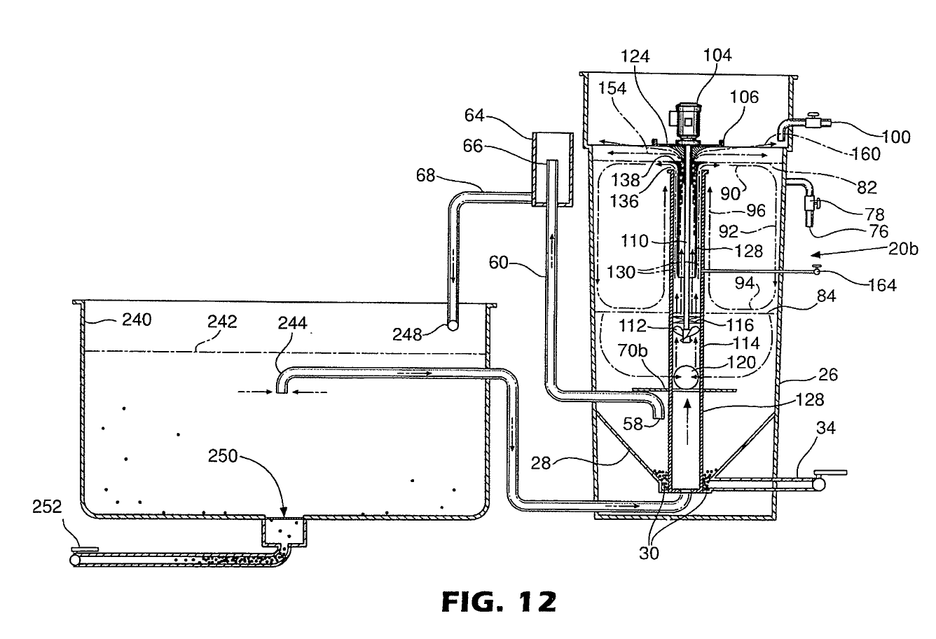

FIG. 12 illustrates an internal pump embodiment in association with aquaculture tank 240 similar to that of FIG. 11 with similar references used. The bio-filter 20b however is similar to that of FIGS. 3 to 6 herein, particularly FIG. 4. It will be apparent that impeller 112 draws in wastewater through inlet 244 as well as recirculating filtrate through aperture 120 in manifold 128.

(9) Bio-Filter with Multiple Disc Diffusers

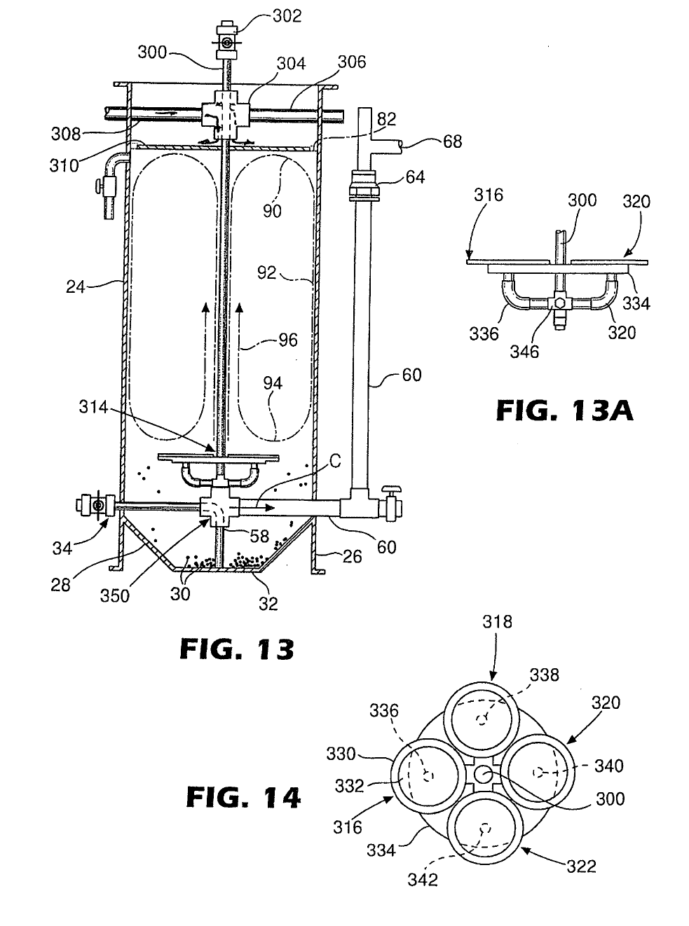

FIGS. 13, 13a and 14 are directed to a bio-filter or bio-reactor with multiple disc diffusers. Like features to those in other embodiments bear similar numerical references. In this embodiment, there is a valved air inlet manifold 300 extending vertically concentric with the tank, the flow of air controlled by valve 302. Inlet manifold 300 is supported by distributor block 304 which is supported by arm 306 in conjunction with a wastewater manifold 308. Air manifold 300 extends through block 304 whereas wastewater manifold 308 extends into block 304 which directs wastewater or liquid downwardly onto circular distributor plate 310, a liquid deflecting means which is supported by air manifold 300. Plate 310 extends outwardly to adjacent the tank wall 24 but provides for a peripheral gap for wastewater to flow into the tank adjacent the wall. At its lower end 314, air inlet manifold 300 is in flow communication with a plurality of air diffusers 316, 318, 320 and 322 as shown also in FIG. 14. Diffusers 316-322 are similarly constructed and each comprises a disc in the form of ring 330 with a perforated rubber membrane 332. The diffusers are supported in plate 334 to which air distributor manifolds 336, 338, 340 and 342 are centrally connected with common junction manifold 346 in flow connection with the air inlet manifold 300. Support 350 provides support for the diffusers as well as itself being effectively supported by valved outlet manifold 34 for particulates and treated fluid outlet manifold 60. The treated fluid inlet 48 in flow communication with treated fluid outlet manifold 60 is within and part of support 350.

In smaller bio-filters up to about 30 inches (0.75 meters), one diffuser disc can be used, but in large filters such as that illustrated in FIG. 13 multiple diffusers are manifolded together to effect the central upwelling of media and filtrate. Plate 310 is important in this embodiment and is located sub-adjacent the surface of the filtrate so that the upwelling of media and filtrate is forced radially outwardly to cause the desired toroidal flow configuration as in other embodiments. The bottom of plate 310 could be contoured as noted with respect to the embodiment of FIG. 9.

In operation the diffusers being centrally located within the tank cause air to rise adjacent to the center causing media and filtrate to rise and thus, in cooperation with plate 310 sets-up a toroidal configuration to the movement of media and pellets shown by flow arrows 90-98 similar to that in other embodiments.

General Comments and Operation

The new design continuously stirs the entire media bed in an energy efficient manner. There are no mechanical moving parts required other than those of the circulation pumps, such as pumps 52 (FIG. 1), 112 (FIG. 3) or 170 (FIG. 7). The circulation pattern of the media bed is toroidal in shape, as shown in the drawings by arrows 90, 92, 94 and 96. The toroidal circulation pattern is achieved by injecting the filtrate at the center surface of the bio-filter or bio-reactor media bed with the water injected designs. Filtrate is injected through an adjustable opening, liquid deflecting means or nozzle which is located just below the surface (a preferable setting being approximately 5 cm). The relatively high velocity, horizontal, radial flow of filtrate forces the surface media, preferably the plastic pellets having a buoyancy less than 1 to flow outward radially (arrows 90) to the outer wall 20 of the filter tank. The filtrate and media then flow down the inside of the wall of the filter (arrows 92) until the media reaches the base 84 of the media bed. At the bottom of the media bed and the media is then drawn radially back toward the center of the bio-filter (arrows 94) and then turns vertically and travels back up the center of the filter to the top (arrows 96) where it begins the rotational cycle again.

Although the media returns to the surface once it reaches the base 84 of the bed, the filtrate continues downward to exit the base of the filter in the external pumped design (FIG. 1), or is drawn down below the media bed to enter the pump inlet at the filter base in the internally re-circulated design (FIG. 3, FIG. 7, FIG. 9). The design therefore is successful in retaining the buoyant media in the surface zone while allowing the filtered liquid to be removed below the media bed without the necessity and use of outlet screens in most embodiments. Additionally the downflow of filtrate still allows the settling and collection of solids in the cone shaped collector 28.

The liquid velocities across the upper surface and down the outer filter wall are beyond the terminal velocity of the buoyant media which entrains the media and causes it to flow with the filtrate. As mentioned previously, the deflectors or distribution plate of the nozzle is adjusted to be slightly below the surface of the media bed. If the inlet is located too shallow or above the media bed surface, the incoming filtrate does not force the media at the central upper surface of the media to flow radially outward and a “dead zone” of static media is created. There is a preferred depth at which the incoming filtrate flow will induce the toroidal affect and stir the entire bed. The deflector or distribution disk is preferably suspended on an adjustable threaded rod so the elevation can be easily fine tuned to optimize stirring. Additionally, the elevation of the media bed itself can be adjusted relative to the deflector or distribution disk by adjusting the external level control pipe sleeve 66 which is located in the media level control chamber 64.

Once the downward media/filtrate flow reaches the interface between the bottom of the media bed and the liquid below the bed, the downward liquid velocity decreases with the increased liquid cross sectional area below the media bed. The fluid velocity, therefore drops below the terminal velocity required to entrain the media. The media, therefore leaves the downward filtrate flow and rises up at the centre of the media bed. The media rises back up the centre to the top of the bed where the cycle is repeated.

The high velocity turbulent flow at the top centre of the bed is sufficient to strip excess bio-film from the media to maintain an optimal thickness for diffusion of nutrients and oxygen. Additionally any media pellets that might accumulate sufficient bio-film to drop out of the bottom of the media bed would be drawn into the intake of a recirculation pump impeller where the extreme turbulence would shear off excess media and return it to the surface of the bed. No secondary shearing devices are needed.

In a fluidized media design, the fluidizing action causes the media particles to hover within a small zone within the filter and do not circulate throughout the filter. With the new stirred design, with each cycle, all of the media particles are brought into the well aerated surface zone of the filter, where oxygen supplies are replenished through contact with the air.

The design is very energy efficient because the pumps can operate with very low or no vertical lift. High flows can be created with low energy axial flow impeller designs (FIGS. 1, 3 & 7) or Simple Air Lift Pumps (FIG. 9).

A “divided” or “split flow” design option provides a simple system to provide both media circulation and supplementary aeration with the use of a single circulating pump or air pump, which can be either internally (FIGS. 3, 9 & 12) or externally mounted FIGS. 1 & 11.

In the split flow design option, the filtrate is injected through adjustable concentric disks (eg. 124 and 136) that divide the flow. A lower subsurface flow 90 can be adjusted to provide just enough flow to stir the bed while an upper aeration flow can be created to provide aeration to supply oxygen for respiration of the micro-organisms in the bio-film. The aeration flow effect is similar to conventional surface splash aerator used for wastewater aeration. Such aerators have high aeration efficiencies as expressed in units of oxygen transferred per horsepower hour. The distribution disks 124 and 136 can be flat but preferably would have contoured surfaces as shown to reduce friction head loss and enhance flow.

In concentrated wastewater streams with high Biochemical Oxygen Demand (BOD), aeration alone may not be sufficient to provide all oxygen required for microbial respiration. Injection of pure oxygen, e.g. through conduit 164, can be a very cost effective way of providing the oxygen needed especially in warm wastewater flows where oxygen solubility is low and surface aerators are less efficient. The divided or split flow design provides the opportunity to efficiently inject pure oxygen without the use of external pressure pumps or diffusers. If the oxygen is injected into the upper aeration flow 160, any supersaturated oxygen would be lost to the atmosphere. Oxygen is preferably injected only into the subsurface flow to minimize outgassing. Supersaturated oxygen levels could be achieved with this method without risk of the supersaturated oxygen outgassing to the atmosphere. A horizontal baffle (272 in FIG. 7) can also be installed at the media surface to help contain non-dissolved oxygen and further prevent outgassing if high injection rates are required. Normally, the high BOD of the wastewater rapidly depletes the oxygen from the wastewater and by the time the media recirculates to the top of the bed, the oxygen is depleted. Since the oxygen is injected via conduit 164 into a relatively large flow of filtrate where it rapidly disperses throughout the filtrate, supersaturated conditions are unlikely to occur so that outgassing is reduced or prevented. Outgassing will only occur if the concentration of oxygen in the liquid is greater than the surrounding atmosphere i.e., in a supersaturated state.

Alternatively, the bio-reactor can be equipped with an air-tight lid (such as seen in FIG. 9) and oxygen can be injected into the upper splash zone above the media bed. The high filtrate flow and splashing at the surface forms small droplets with a very high surface area. Exposure of the droplets to a pure oxygen environment result in excellent oxygen transfer efficiency. This design would rely entirely on injected pure oxygen for respiration since there is no zone where the filtrate is exposed to ambient air. The two phase aeration and oxygen injection design described previously take advantages of oxygen in the surrounding atmosphere so that less supplemental pure oxygen is required.

The central radial injection nozzle, i.e., liquid deflecting means, is much simpler than the distribution devices required in fluidized designs where the filtrate must be evenly distributed over the entire surface of the media bed in order to create a uniform downward velocity. As the filter size increases, the surface area of the filter becomes larger and the structures required to achieve uniform distribution become more complex. Use of perforated distribution plates in such designs is also a problem because the large surface area requires a large number of uniformly distributed holes. To divide the flow over the large number of holes requires relatively small orifices which are prone to bio-fouling so that regularly cleaning or automated cleaning systems are required. Scale-up of the new central stirring nozzle is very simple and inexpensive and the large opening of the nozzle prevents bio-fouling.

Another advantage of the new stirred bed design is that there is very little expansion of the media bed when the filter is started up. That is the depth of the media bed when the filter is operating is only slightly greater (<20%) than the depth of the media bed when the filter is turned off. Fluidized beds inherently have significant expansion of the bed when turned on (up to 100%). The stirred bed therefore can accommodate significantly more media per unit volume of filter size since volume is not wasted in allowing for expansion as in a fluidized design.

Fluidized designs also are usually tall and narrow cylinders (small cross section), so that the relatively high fluidization velocities can be achieved with reasonable filtrate flow rates. This increases pump head requirements but also limits the flexibility of designing lower profile bio-filters where available height is limited. The new stirred design is not limited to narrow tall designs and works equally well with shallow wide tank designs.

The air stirred version, FIGS. 13 and 14, use diffused air injected below the center of the media bed to stir the media. A column of air/media/filtrate mixture rises up the center of the media bed. At the surface of the media bed, the mixture encounters the circular distribution plate and is forced to flow radially outward to the periphery of the filter. The circulation flow and dynamics of the stirring are otherwise similar to the water injected designs previously discussed. The filtrate entering the filter is injected through a central opening on top of the distribution plate and the filtrate flows radially across the top surface towards the filter tank periphery. This results in an even distribution and mixing of the incoming filtrate with the filter media.

Configuring Options

As shown in the drawings, they show various possible configurations of filters with external large pumped supplies of filtrate (FIG. 2) and both internal recirculated (FIGS. 3 and 7) and externally recirculated (FIG. 1).

Treating concentrated wastewater flows sufficiently to enable discharge usually requires long residence times and relative small wastewater flows per unit of filter volume. The flows required to stir the media bed are much greater than the filtrate flows. An internal or external pumped recirculation loop (FIGS. 1, 3, 4, 5 & 8) is therefore used to provide circulation flows sufficient for stirring and supplemental aeration if required. The circulating pump can be of any energy efficient design. Axial flow pumps with surface mounted motors (FIG. 3) or submersible motors (FIG. 7) and Air Lift Pumps (FIG. 9) are particularly well suited to this application because they are capable of circulating large flows of filtrate in low lift applications with very low energy consumption. Since the filtrate is circulated with essentially no vertical lift, the pump only has to overcome friction losses from the pipe and fittings. Also as noted above, air diffusers provide an effect means in co-operation with a distributing plate adjacent the top surface of creating a toroidal configuration to the filtrate and media.

Bio-filters are used in typical aquaculture operations to culture nitrifying bacteria which remove toxic ammonia from culture water to enable re-use of the water. Total ammonia levels generally are maintained below 1 mg/l which is very dilute relative to municipal or industrial wastewaters. With such dilute wastewaters, only short residence times are required to treat the wastewater. To maintain the acceptably low concentrations, therefore, high flow rates of wastewater must be pumped through the filter. The wastewater flows would normally be in the range required for successfully stirring the media bed. The filtrate would be pumped in through the previously described inlet to stir the bed (FIG. 11). A variation of this design is shown in FIG. 12 where the internal axial flow pump 112 in the bio-filter is connected to the clear-water effluent pipe 244 from the fish tank. A valve could be located on the fish tank line to determine how much filtrate is drawn from the tank and how much filtrate is re-circulated within the filter via inlet 32.

A simple variation of the filter is shown in FIG. 8 which is suitable to supplement the filtration capacity in existing wastewater treatment reservoirs or ponds. The filter would be equipped with a flotation collar 190 or alternately could be suspended in the pond on adjustable columns set into the pond bottom. The filter pump inlet 196 would be extended to draw wastewater from the base of the pond and pass it down though the filter media bed to exit at the outer base of the filter at 192. The filter would operate in a zero lift situation so the circulation would be very efficient. This design would provide increased capacity for facilities with ponds without having to install filtration in line before or after the pond to increase capacity. The filter would not be equipped with a cone shaped bottom for solids collection.

Although various embodiments are shown and described above, other modifications and variations will be apparent to those skilled in the art and the invention includes those modifications and variations which fall within the scope of the appended claims.

What is claimed is:

1. A bio-filter having a downflow self-cleaning configuration comprising liquid deflecting means generally concentric with a cylindrical tank having a bed of buoyant media pellets therein, means for directing liquid to be treated at said deflecting means, said deflecting means located adjacent the upper surface of liquid in the tank such that liquid impinging said deflecting means is directed radially outwardly at the upper surface of the liquid towards an inner wall surface of the tank causing media pellets to move radially outwardly towards said inner wall surface, and downwardly adjacent the inner wall surface, the pellets being further caused to move inwardly toward the center of the tank and upwardly adjacent the center of the tank, the liquid circulating within the tank causing the media pellets to move in a toroidal configuration stirring the buoyant media pellets within the tank.

2. The bio-filter of claim 1 further comprising means for vertically adjustably supporting said deflecting means within said tank such that liquid ejected from the deflecting means is subsurface the upper level of liquid in the tank.

3. The bio-filter of claim 1 further comprising means wherein the deflecting means is in flow communication with liquid in said tank to be recirculated for treatment.

4. The bio-filter of claim 1 further comprising means wherein the deflecting means is in flow communication with a wastewater supply manifold.

5. The bio-filter of claim 1 further comprising a recirculation manifold supported generally concentric with said tank, a motor driven impeller supported within said recirculating manifold, and means for adjustably supporting said deflecting means above an upper end of said recirculation manifold.

6. The bio-filter of claim 5 wherein said recirculation manifold comprises a peripheral wall and a concentric flow divider therein, wherein a portion of the liquid in the recirculation manifold is directed upwardly in a first flow path within the divider and a second portion of the liquid is directed upwardly in a second flow path between the divider and the peripheral wall of the recirculation manifold, said deflecting means being above the second flow path to provide for the toroidal fluid circulation, second means above said deflecting means for deflecting liquid in said first flow path outwardly toward said tank wall and above the level of liquid in the tank to cause aeration of liquid from said first path.

7. The bio-filter of claim 6 further comprising means adapted for adjusting the height of the deflecting means relative to the liquid level in said tank.

8. The bio-filter of claim 6 further comprising means adapted for adjusting the height of said second means for deflecting liquid in said first low path relative to the liquid in said tank.

9. The bio-filter of claim 1 further comprising means for controlling the level of liquid in the tank.

10. The bio-filter of claim 9 further comprising means for withdrawing treated liquid from the tank and means for adding wastewater to the tank to maintain a desired level of liquid in the tank.

11. The bio-filter of claim 5 wherein said motor driven impeller is part of a submersible pump and within the recirculation manifold.

12. The bio-filter of claim 10 wherein said recirculation manifold comprises a peripheral wall and a concentric flow divider therein, wherein a first portion of the liquid in the recirculation manifold is directed upwardly in a first flow path within the divider and a second portion of the liquid in the manifold is directed upwardly in a second flow path between the divider and the peripheral wall of the recirculation manifold, said deflecting means being above the second flow path to provide for the toroidal fluid circulation, and second means above said deflecting means for deflecting liquid in said first flow path outwardly toward said tank wall and above the level of liquid in the tank to cause aeration of liquid from said first path.

13. The bio-filter of claim 12 further comprising means adapted for adjusting the height of the deflecting means relative to the liquid level in said tank.

14. The bio-filter of claim 12 further comprising means adapted for adjusting the height of said second means for deflecting liquid in said first flow path relative to the liquid in said tank.

15. The bio-filter of claim 1 wherein said tank is supported by pontoons for floating in a reservoir containing liquid to be treated.

16. The bio-filter of claim 15 wherein said tank comprises a concentric liquid intake manifold with said deflecting means supported on a liquid intake manifold pump means within said intake manifold and means adapted for allowing treated liquid to return to the reservoir, said liquid intake manifold having an inlet for reservoir liquid to be treated extending below said means for allowing return of treated liquid to said reservoir.

17. The bio-filter of claim 16 wherein said means adapted for allowing the return of treated liquid comprises a peripheral gap defined between a bottom surface of said tank attached to said reservoir liquid intake manifold and a peripheral wall of the tank.

18. The bio-filter of claim 1 wherein said tank includes a concentric recirculation manifold supported within the tank, and air pump means within said recirculation manifold, said deflecting means defined by a disk adjustably supported by and spaced above an upper end of said recirculation manifold adjacent the surface of liquid in the tank, said recirculation manifold including an inlet and said air pump being adapted to recirculate liquid in said tank and cause the media pellets to flow in the toroidal configuration.

19. The bio-filter of claim 18 further comprising means adapted to withdraw treated liquid from the tank.

20. A filtration system comprising the bio-filter of claim 19.

21. The bio-filter of claim 1 wherein said deflecting means is supported by a wastewater manifold concentric with said tank, said deflecting means comprising a contoured disk adjustably supported upon said manifold and in cooperation with a plate also secured to a wastewater manifold defining nozzle means for ejecting water radially adjacent the upper surface of said liquid.

22. The bio-filter according to claim 21 wherein said wastewater manifold includes apertures above said plate for spraying liquid radially outwardly to permit aeration of a portion of wastewater flow in said manifold, and means adapted for injection of oxygen into said manifold.

23. The system of claim 21 further comprising means adapted to collect particulate material in the bio-filter tank and for periodic removal thereof.

24. In an aquaculture system comprising a fish tank and bio-filter, said bio-filter having a downflow self-cleaning configuration comprising a liquid deflecting means generally concentric with a cylindrical bio-filter tank having a bed of buoyant media pellets therein, means for directing liquid to be treated at said deflecting means, said deflecting means located adjacent an upper surface of liquid in the tank such that said liquid impinges on said deflecting means and is directed radially outwardly at the upper surface of the liquid towards an inner wall surface of the bio-filter tank causing media pellets adjacent said upper surface to move radially outwardly toward said inner wall surface and downwardly adjacent the inner wall surface, the pellets being further caused to move inwardly toward the center of the tank and upwardly adjacent the center of the bio-filter tank, the liquid circulating within the bio-filter tank causing media pellets to move in a toroidal flow configuration stirring the buoyant media pellets within the bio-filter tank, first flow communication means for withdrawing liquid to be treated from the fish tank and directing said liquid to said deflecting means and second flow communication means adapted to permit treated liquid to flow from said bio-filter tank to said fish tank.

25. The system of claim 24 further comprising means adapted to collect particulate material in said fish tank and for periodic removal thereof.

26. In a bio-filter having a tank with a peripheral wall and longitudinal axis, apparatus configured to impart a toroidal flow configuration to filtrate within the tank and cause media pellets in a media bed in the tank to be stirred and move in said configuration to self clean said pellets of bio-film, said apparatus comprising: deflecting means adjacent the surface of the filtrate to peripherally direct flow of filtrate radially outwardly toward said tank wall, said wall redirecting said filtrate and media pellets downwardly along the wall, said media pellets moving inwardly to the centre of the tank adjacent the bottom of the media bed and upwardly to the surface adjacent the axis of the tank to define said toroidal configuration; and
means for creating flow of liquid to said deflecting means.

27. The bio-filter of claim 26 wherein the means creating flow of liquid is at least one air diffuser located below said media bed and creating air flow upwardly adjacent said axis to cause filtrate to rise and impinge on said deflecting means.

28. The bio-filter of claim 26 wherein the deflecting means is a nozzle adjacent the surface of said filtrate to peripherally direct said filtrate outwardly, and wherein said means for creating said flow comprises means for supplying filtrate under pressure to said nozzle.

29. In a method of treating liquid in a bio-filter comprising a tank having a peripheral wall and longitudinal axis, said tank containing said liquid and a bed of buoyant media pellets, the step of:
directing movement of liquid radially outwardly from adjacent the axis of the tank along the upper surface of said media bed resulting in the liquid also moving downwardly along the wall and thereby causing media pellets to move outwardly and down the wall of the tank, said buoyant pellets thereafter moving radially inwardly at the bottom of said media bed and upwardly adjacent the axis of said tank to define a toroidal flow configuration to the media in the bed as a result of the movement of liquid radially outwardly and downwardly.

30. The method of claim 29 wherein said directing movement of liquid is caused by liquid being ejected from a nozzle under pressure radially along the upper surface of the media bed in the liquid.

31. The method of claim 29 wherein said directing movement of liquid is caused by a deflection plate adjacent said liquid surface extending radially outwardly towards said tank wall, said plate operating in co-operation with at least one air generation means adjacent said tank axis and below the bottom of said media bed wherein said liquid is caused to flow upwardly adjacent the axis and be deflected outwardly along the surface of the bed to create the toroidal flow configuration of the pellet media.
